(19) United States
(12) Patent Application Publication
(10) Pub. No.: US 2022/0197018 A1
(43) Pub. Date: Jun. 23, 2022

(54) Optical Instrument and Method for Use

(71) Applicants: University of Washington, Seattle, WA (US); The Board of Trustees of the Leland Stanford Junior University, Stanford, CA (US)

(72) Inventors: Ramkumar Sabesan, Seattle, WA (US); Vimal Prabhu Pandiyan, Seattle, WA (US); Daniel Palanker, Seattle, WA (US)

(21) Appl. No.: 17/605,182

(22) PCT Filed: Apr. 25, 2020
(86) PCT No.: PCT/US2020/029984

§ 371 Date: Oct. 20, 2021

Related U.S. Application Data
(60) Provisional application No. 62/839,072, filed on Apr. 26, 2019.

(51) Int. Cl. G02B 26/10; A61B 3/10; G02B 21/00; G01B 9/02; G01B 9/02091; A61B 5/00

(52) U.S. Cl. CPC G02B 26/101; A61B 3/102; A61B 5/0066; G01B 9/02044; G01B 9/02091; G02B 21/0012

(57) ABSTRACT

An optical instrument includes a first light source configured to generate a broadband light; an optical module configured to collimate the broadband light and focus the broadband light into a line; a beam splitter configured to split the broadband light into a sample beam and a reference beam and configured to combine the reference beam with the sample beam to form an interference beam; a control system configured to scan the sample beam on a retina of a subject along an axis that is substantially perpendicular to the sample beam; a second light source configured to stimulate the retina with a visible light to induce a physical change within the retina such that the sample beam is altered by the physical change; an image sensor; and a dispersive element configured to receive the interference beam from the beam splitter and to disperse the interference beam onto the image sensor.

### CROSS-REFERENCE TO RELATED APPLICATION

This application claims priority to U.S. Provisional Patent Application No. 62/839,072, filed on Apr. 26, 2019, the entire contents of which are incorporated herein by reference.

### FEDERAL FUNDING STATEMENT

This invention was made with government support under Grant Nos. R21 EY027941 and U01 EY025501, awarded by the National Eye Institute. The government has certain rights in the invention.

Also incorporated by reference herein is “The optoretinogram reveals how human photoreceptors deform in response to light,” Vimal Prabhu Pandiyan, Aiden Maloney Bertelli, James Kuchenbecker, Kevin C Boyle, Tong Ling, B. Hyle Park, Austin Roorda, Daniel Palanker, Ramkumar Sabesan, bioRxiv 2020.01.18.911339; doi: https://doi.org/10.1101/2020.01.18.911339.

### BACKGROUND

Retinal diseases are a leading cause of blindness and other vision disorders. To identify and treat retinal diseases, instruments capable of imaging both the structure of the retina and the retina's response to visual stimuli are important. Both high spatial resolution and high temporal resolution are important for obtaining useful information about the retina. Conventional optical instruments for imaging the structure and/or response of the retina are often lacking in high spatial resolution, high temporal resolution, and/or good signal to noise ratio.

### SUMMARY

In a first aspect of the disclosure, an optical instrument comprises: a first light source configured to generate a broadband light; an optical module configured to collimate the broadband light and focus the broadband light into a line; a beam splitter configured to split the broadband light into a sample beam and a reference beam and configured to combine the reference beam with the sample beam to form an interference beam; a control system configured to scan the sample beam on a retina of a subject along an axis that is substantially perpendicular to the sample beam; a second light source configured to stimulate the retina with a visible light to induce a physical change within the retina such that the sample beam is altered by the physical change; an image sensor; and a dispersive element configured to receive the interference beam from the beam splitter and to disperse the interference beam onto the image sensor.

In a second aspect of the disclosure, a method of operating an optical instrument comprises: generating a broadband light that has a shape of a line; splitting the broadband light into a sample beam and a reference beam; scanning the sample beam on a retina of a subject along an axis that is substantially perpendicular to the sample beam; stimulating the retina with a visible light to induce a physical change within the retina such that the sample beam is altered by the physical change; combining the reference beam with the sample beam to form an interference beam; and dispersing the interference beam onto an image sensor.

In a third aspect of the disclosure, a non-transitory computer readable medium stores instructions that, when executed by one or more processors of an optical instrument, cause the optical instrument to perform functions comprising: generating a broadband light that has a shape of a line; splitting the broadband light into a sample beam and a reference beam; scanning the sample beam on a retina of a subject along an axis that is substantially perpendicular to the sample beam; stimulating the retina with a visible light to induce a physical change within the retina such that the sample beam is altered by the physical change; combining the reference beam with the sample beam to form an interference beam; and dispersing the interference beam onto an image sensor.

When the term “substantially” or “about” is used herein, it is meant that the recited characteristic, parameter, or value need not be achieved exactly, but that deviations or variations, including, for example, tolerances, measurement error, measurement accuracy limitations, and other factors known to those of skill in the art may occur in amounts that do not preclude the effect the characteristic was intended to provide. In some examples disclosed herein, “substantially” or “about” means within +/- 0-5% of the recited value.

These, as well as other aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate the invention by way of example only and, as such, that numerous variations are possible.

### BRIEF DESCRIPTION OF THE DRAWINGS

FIG. 1 is a schematic diagram of an optical instrument, according to an example embodiment.

FIG. 2 is a block diagram of a computing system, according to an example embodiment.

FIG. 3 is a schematic diagram of captured images, according to an example embodiment.

FIG. 4 is a schematic diagram of transformed images, according to an example embodiment.

FIG. 5 is schematic diagram of imaging techniques, according to an example embodiment.

FIG. 6 is a block diagram of a method, according to an example embodiment.

### DETAILED DESCRIPTION

As noted above, optical instruments configured for imaging a retina with high spatial resolution, high temporal resolution, and high signal to noise ratio are needed. Examples of such optical instruments and methods for using them are discussed in the present disclosure.

Within examples, an optical instrument includes a first light source configured to generate a broadband light and an optical module configured to collimate the broadband light and focus the broadband light into a line. The optical instrument also includes a beam splitter configured to split the broadband light into a sample beam and a reference beam and configured to combine the reference beam with the sample beam to form an interference beam. The optical instrument also includes a control system configured to scan the sample beam on a retina of a subject along an axis that is substantially perpendicular to the sample beam and a second light source configured to stimulate the retina with a visible light to induce a physical change within the retina such that the sample beam is altered by the physical change. The optical instrument also includes an image sensor and a dispersive element configured to receive the interference beam from the beam splitter and to disperse the interference beam onto the image sensor.

Embodiments disclosed herein can provide improved spatial and temporal resolution when compared to conventional instruments. Because the sample beam disclosed herein is generally a line-shaped beam, scanning of the sample beam over a two-dimensional area of the retina is generally only required over only one axis, which can greatly improve the amount of image data that can be captured per unit time. Since a subject's eye (e.g., retina) will generally move somewhat over time, accurate imaging will generally require that the entire region of interest of the retina is imaged relatively quickly before the subject's eye has had a chance to move significantly. Using a two dimensional image sensor that can operate at frame rates ranging from 2,500-16,000 Hz, or at even higher frame rates, can be useful in achieving high spatial and temporal resolution. Using these imaging techniques, various phenomena of the subject's eye can be analyzed across a range of spatial and temporal resolution with respect to decay time, latency of response onset, and duration of response, etc. and ranging from single cells to a collection of many cells.

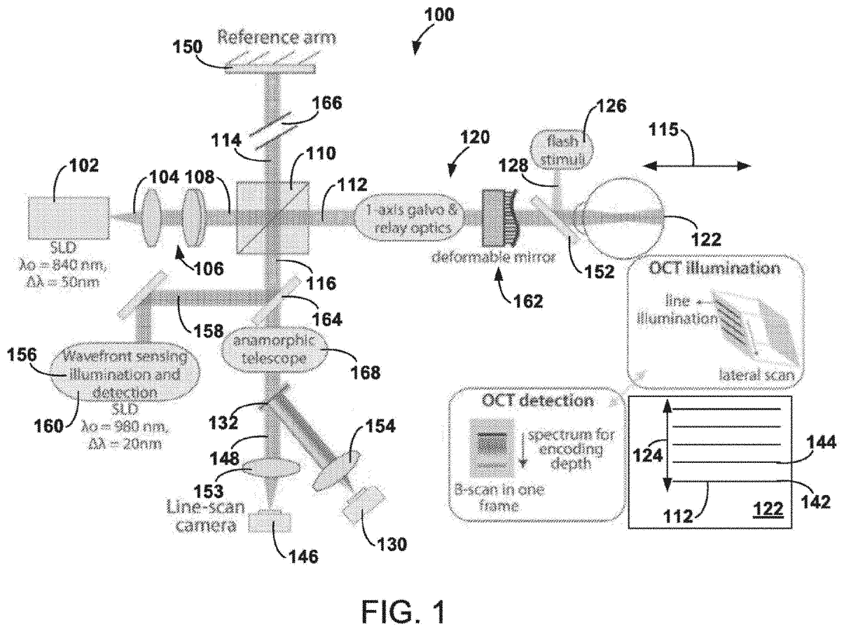

FIG. 1 is a schematic diagram of an optical instrument 100. The optical instrument 100 includes a first light source 102 configured to generate a broadband light 104 and an optical module 106 configured to collimate the broadband light 104 and focus the broadband light 104 into a line 108 (e.g., having a length ranging from 400 pm to 500 pm on the retina 122). The optical instrument 100 also includes a beam splitter 110 configured to split the broadband light 104 into a sample beam 112 and a reference beam 114 and configured to combine the reference beam 114 with the sample beam 112 to form an interference beam 116. The optical instrument 100 also includes a control system 120 configured to scan the sample beam 112 on the retina 122 of a subject along an axis 124 that is substantially perpendicular to the sample beam 112. The optical instrument 100 also includes a second light source 126 configured to stimulate the retina 122 with a visible light 128 to induce a physical change within the retina 122 such that the sample beam 112 is altered by the physical change. The optical instrument 100 also includes an image sensor 130 and a dispersive element 132 configured to receive the interference beam 116 from the beam splitter 110 and to disperse the interference beam 116 onto the image sensor 130.

The optical instrument 100 and the recorded light-induced optical changes from the retina 122 can be referred to as an optoretinogram.

The first light source 102 can include a super-luminescent diode or a supercontinuum source, but other examples are possible.

The broadband light 104 can have a center wavelength of 840 nanometers (nm) and/or a full width half maximum (FWHM) within a range of 15 nm to 150 nm, (e.g., 50 nm). When leaving the first light source 102, the broadband light 104 is generally not collimated or focused.

The optical module 106 includes a positive powered lens or a mirror that collimates the broadband light 104 and a cylindrical lens that focuses the broadband light into the line 108. Other examples are possible.

The beam splitter 110 generally takes the form of two triangular prisms that are adhered to each other to form a cube, or a plate beam splitter. The discontinuity between the two prisms performs the beam splitting function. Thus, the beam splitter 110 splits the line-shaped broadband light 104 into the sample beam 112 and the reference beam 114. The reference beam 114 travels from the beam splitter 110, through the optical module 166, reflects off the mirror 150, travels back through the optical module 166, and back to the beam splitter 110. The sample beam 112 is scanned by the control system 120 and/or formed by the deformable mirror 162, and transmits through the filter 152 onto the retina 122. The sample beam 112 reflects and/or scatters off of the retina 122, travels through the filter 152, and back to the beam splitter 110. The beam splitter 110 combines the reference beam 114 with the sample beam 112 to form the interference beam 116. Thus, the interference beam 116 constitutes a superposition of the reference beam 114 and the sample beam 112, and the optical instrument 100 can operate as an interferometer.

The optical module 166 is configured to maintain collimation and/or coherence of the reference beam 114. The distance between the beam splitter 110 and the mirror 150 can be several meters are more, and the collimation and/or coherence of the reference beam 114 can be degraded over such distances without compensation. Thus, the optical module 166 can include lenses and/or mirror-based telescopes that maintain collimation and/or coherence of the reference beam 114.

The mirror 150 is configured to reflect the reference beam 114 back to the beam splitter 110. The mirror 150 generally has a reflectance that is substantially equal to 100% over the visible and infrared spectrum, but other examples are possible.

The control system 120 can include a galvanometer that can scan (e.g., deflect) the sample beam 112 along an axis 124 on the retina 122 (inset at the bottom right of FIG. 1). As shown, the axis 124 is perpendicular to the sample beam 112. For example, the control system 120 can scan the sample beam 112 such that the sample beam 112 illuminates a line-shaped position 142 on the retina 122, and then illuminates a line-shaped position 144 on the retina 122, and so on. The control system 120 can also control the deformable mirror 162, as described in more detail below. The control system 120 generally includes hardware and/or software configured to facilitate performance of the functions attributed to the control system 120 herein.

The sample beam arm of the optical instrument 100 can also include an optical module similar to the optical module 166 that is configured to maintain collimation and/or coherence of the sample beam 112 (referred to as “relay optics” in FIG. 1).

The second light source 126 can take the form of a light emitting diode, but other examples are possible. The visible light 128 can have a full width half maximum (FWHM) within a range of 10 nm to 50 nm and have a center wavelength of 528 nm, 660 nm, or 470 nm, for example. The visible light 128 could generally have any center wavelength within the visible light spectrum. The visible light 128 is directed upon the retina 122 by the filter 152. The visible light 128 can induce physical changes in the retina 122 such as movement and/or changes in size or shape of retinal neurons in any of the three dimensions. In some examples, the physical change in the retina 122 can include a change in refractive index and/or optical path length of one or more retinal neurons, a change in electrical activity in one or more retinal neurons, and/or a change in constituents of one or more retinal neurons. In some examples, the visible light 128 consists of one or more pulses of light having varying or constant pulse widths (e.g., 500 μs to 100 ms) and/or intensities, but other examples are possible.

The filter 152 is configured to direct the visible light 128 to the retina 122 and to transmit the sample beam 112 from the retina 122 back to the beam splitter 110. Thus, the filter 152 has a non-zero transmissivity for at least infrared light.

The image sensor 130 typically takes the form of a complementary metal-oxide-semiconductor (CMOS) or charge-coupled device (CCD) image sensor (e.g., a high speed camera).

The dispersive element 132 is typically a diffraction grating (e.g., transmissive or reflective), but a prism could be used as well. Other examples are possible. The dispersive element 132 is configured to receive the interference beam 116 from the beam splitter 110 (e.g., from the optical module 164) and to diffract the interference beam 116 onto the image sensor 130. That is, dispersive element 132 disperses the interference beam 116 such that varying spectral components of the interference beam 116 are distinguishable (e.g., positioned on respective lines/portions of the image sensor 130).

The image sensor 146 (e.g., a line scan camera) is configured to capture a substantially one-dimensional image representing a zero-order portion 148 of the interference beam 116 that passes through the dispersive element 132 without being diffracted. When the image sensor 146 is being operated, the reference beam 114 is blocked from the beam splitter 110. Thus, in this example, the interference beam 116 is substantially the same as the sample beam 112 that returns from the retina 122. The zero-order portion 148 of the interference beam 116 is a signal that represents a portion of the sample beam 112 that is back-scattered from the retina 122. The one-dimensional image represents a line-shaped portion of a surface of the retina 122 that is illuminated by the sample beam 112 (e.g., the portion of the retina 122 at position 142). The image sensor 146 can capture one-dimensional images corresponding respectively to various positions on the retina 122 along the axis 124, for example. These one-dimensional images can be pieced together to form a two-dimensional image representing an exposed surface of the retina 122 (e.g, before, during, and/or after stimulation by the visible light 128).

The optical module 153 is configured to adjust a spatial resolution of the zero-order portion 148 and/or focus the zero-order portion 148 so that the area of the image sensor 146 can be efficiently used. The optical module 153 can include one or more lenses and/or mirrors.

The optical module 154 is configured to modify the interference beam 116 after the interference beam 116 has been dispersed by the dispersive element 132 to adjust spatial resolution of the interference beam 116 and/or adjust spectral resolution of the interference beam 116 so that the area of the image sensor 130 can be efficiently used. The optical module 154 can include one or more lenses and/or mirrors and can also be used to focus the interference beam 116 after the interference beam 116 has been dispersed by the dispersive element 132.

The optical module 168 (e.g., an anamorphic telescope), including one or more lenses and/or mirrors, is configured to compress or stretch the interference beam 116 before the interference beam 116 has been dispersed by the dispersive element 132. The optical module 168 typically will include two cylindrical lenses having longitudinal axes that are parallel to each other but rotated at 90 degrees with respect to each other.

The optical instrument 100 also includes a third light source 156 configured to generate a third light 158. The third light source 156 could be an LED, but other examples are possible. The third light 158 can have a center wavelength of 970 nm and a FWHM of 10-30 nm (e.g., 20 nm), but other examples are possible. The optical instrument 100 also includes a wavefront sensor 160 and a second optical module 164 including one or more mirrors and/or lenses configured to direct the third light 158 from the third light source 156 to the beam splitter 110 and from the beam splitter 110 back to the wavefront sensor 160. The beam splitter 110 is further configured to direct the third light 158 to the control system 120. The wavefront sensor 160 is configured to detect optical aberrations of an eye of the subject by analyzing the third light 158 that returns from the retina 122. The control system 120 is configured to control the deformable mirror 162 to form the sample beam 112 on the retina 122 based on the optical aberrations of the eye, (e.g., to compensate for the aberrations of the eye).

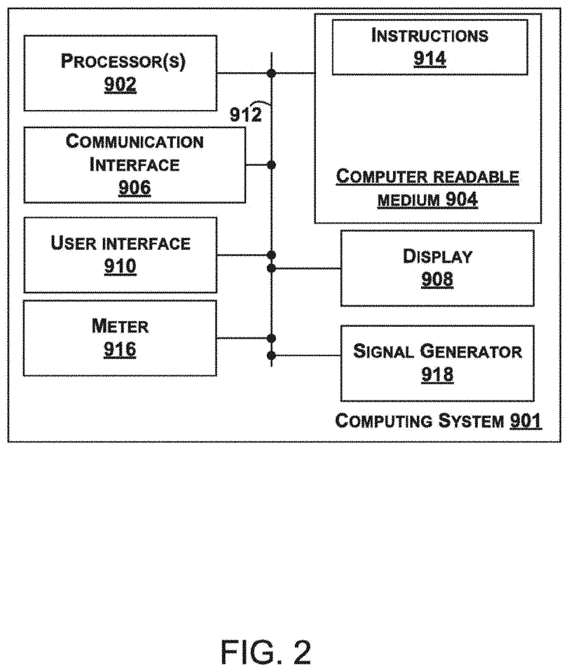

FIG. 2 shows the computing system 901. The computing system 901 includes one or more processors 902, a non-transitory computer readable medium 904, a communication interface 906, a display 908, and a user interface 910. Components of the computing system 901 are linked together by a system bus, network, or other connection mechanism 912.

The one or more processors 902 can be any type of processor(s), such as a microprocessor, a digital signal processor, a multicore processor, etc., coupled to the non-transitory computer readable medium 904.

The non-transitory computer readable medium 904 can be any type of memory, such as volatile memory like random access memory (RAM), dynamic random access memory (DRAM), static random access memory (SRAM), or non-volatile memory like read-only memory (ROM), flash memory, magnetic or optical disks, or compact-disc read-only memory (CD-ROM), among other devices used to store data or programs on a temporary or permanent basis.

Additionally, the non-transitory computer readable medium 904 can be configured to store instructions 914. The instructions 914 are executable by the one or more processors 902 to cause the computing system 901 to perform any of the functions or methods described herein.

The communication interface 906 can include hardware to enable communication within the computing system 901 and/or between the computing system 901 and one or more other devices. The hardware can include transmitters, receivers, and antennas, for example. The communication interface 906 can be configured to facilitate communication with one or more other devices, in accordance with one or more wired or wireless communication protocols. For example, the communication interface 906 can be configured to facilitate wireless data communication for the computing system 901 according to one or more wireless communication standards, such as one or more Institute of Electrical and Electronics Engineers (IEEE) 801.11 standards, ZigBee standards, Bluetooth standards, etc. As another example, the communication interface 906 can be configured to facilitate wired data communication with one or more other devices.

The display 908 can be any type of display component configured to display data. As one example, the display 908 can include a touchscreen display. As another example, the display 908 can include a flat-panel display, such as a liquid-crystal display (LCD) or a light-emitting diode (LED) display.

The user interface 910 can include one or more pieces of hardware used to provide data and control signals to the computing system 901. For instance, the user interface 910 can include a mouse or a pointing device, a keyboard or a keypad, a microphone, a touchpad, or a touchscreen, among other possible types of user input devices. Generally, the user interface 910 can enable an operator to interact with a graphical user interface (GUI) provided by the computing system 901 (e.g., displayed by the display 908).

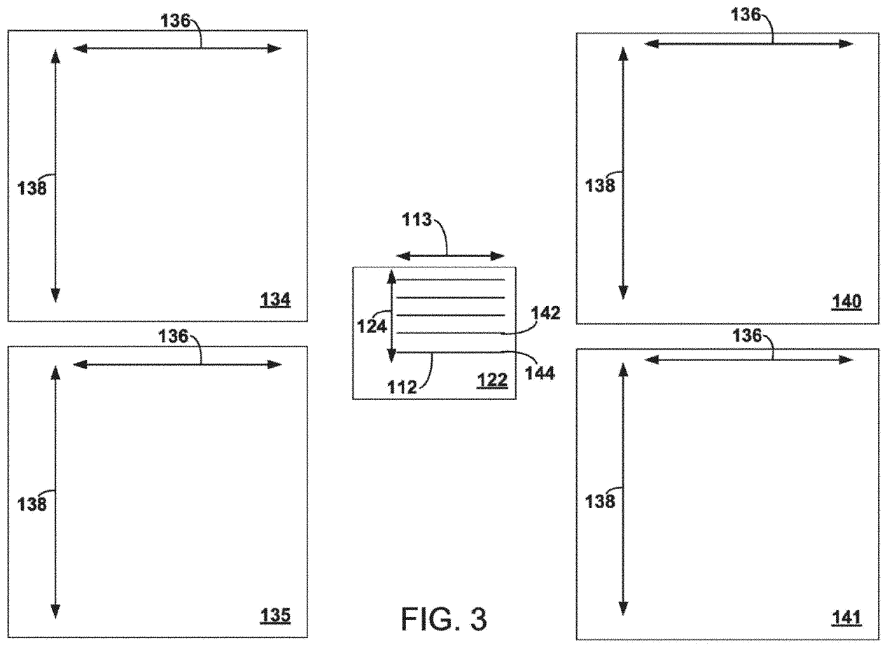

FIG. 3 is a schematic diagram of captured images 134, 135, 140, and 141.

The image sensor 130 is configured to capture a wavelength space image 134 of the interference beam 116 after the interference beam 116 has been dispersed by the dispersive element 132. The wavelength space image 134 is defined by an axis 136 that corresponds to a length 113 of the sample beam 112 and an axis 138 that corresponds to wavelengths of the sample beam 112. That is, wavelengths of the interference beam 116 are dispersed along the axis 138 in order of increasing or decreasing wavelength. The wavelength space image 134 corresponds to the position 142 on the retina 122 along the axis 124. Thus, the wavelength space image 134 corresponds to a cross section or “slice” of the retina 122 corresponding to a plane defined by the sample beam 112 at the position 142 being extended into the retina 122, with the varying wavelengths of the interference beam 116 being a proxy for a depth 115 into the retina 122, as explained further below.

The image sensor 130 is also configured to capture a wavelength space image 140 of the interference beam 116 after the interference beam 116 has been dispersed by the dispersive element 132. The wavelength space image 140 is also defined by the axis 136 and the axis 138. Similar to the wavelength space image 134, in the wavelength space image 140, wavelengths of the interference beam 116 are dispersed along the axis 138 in order of increasing or decreasing wavelength. The wavelength space image 140 corresponds to the position 144 on the retina 122 along the axis 124. Thus, the wavelength space image 140 corresponds to a cross section or “slice” of the retina 122 corresponding to a plane defined by the sample beam 112 at the position 144 being extended into the retina 122.

In some embodiments, the image sensor 130 captures additional wavelength space images 135 and 141 subsequent to capturing the wavelength space images 134 and 140 and/or after the retina 122 is stimulated with the visible light 128. In this context, the image sensor 130 can capture the wavelength space image 135 of the interference beam 116 after the interference beam 116 has been dispersed by the dispersive element 132. The wavelength space image 135 is also defined by the axis 136 and the axis 138. The wavelength space image 135 corresponds to the position 142 on the retina 122 along the axis 124. Thus, the wavelength space image 135 corresponds to a cross section or “slice” of the retina 122 corresponding to a plane defined by the sample beam 112 at the position 142 being extended into the retina 122, after the visible light 128 stimulates the retina 122. Thus, the wavelength space image 135 can be compared to the wavelength space image 134 to determine an effect of the visible light 128 at the position 142.

The image sensor 130 can also capture the wavelength space image 141 of the interference beam 116 after the interference beam 116 has been dispersed by the dispersive element 132. The wavelength space image 141 is also defined by the axis 136 and the axis 138. The wavelength space image 141 corresponds to the position 144 on the retina 122 along the axis 124. Thus, the wavelength space image 141 corresponds to a cross section or “slice” of the retina 122 corresponding to a plane defined by the sample beam 112 at the position 144 being extended into the retina 122, after the visible light 128 stimulates the retina 122. Thus, the wavelength space image 141 can be compared to the wavelength space image 140 to determine an effect of the visible light 128 at the position 144.

In some embodiments, the sample beam 112 remains at the position 142 while image data is captured over time. For example, the wavelength space image 135 can be captured (e.g., immediately) after the wavelength space image 134 is captured without scanning the sample beam 112 between capture of the wavelength space image 134 and capture of the wavelength space image 135. This can allow for high temporal resolution scans of one particular cross-sectional area of the retina 122. Such wavelength space images can be transformed into corresponding depth space images that depict signal intensity or signal phase as well, as described below. This technique can also be applied to volumetric scans.

In additional embodiments, the computing system 901 can transform the wavelength space images 134, 140, 135, and 141 into depth space images, as described below.

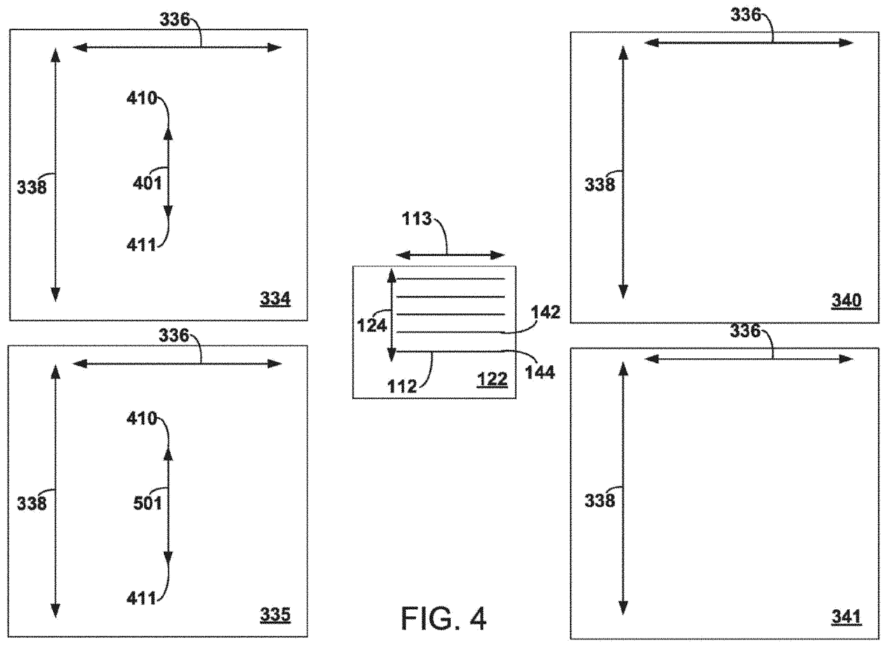

Referring to FIGS. 3 and 4, the computing system 901 can transform the wavelength space image 134 to generate a depth space image 334 comprising a first plurality of pixel values. For example, the computing system 901 can perform a Fourier transform that maps the wavelength space to a depth space, the depth space referring to a depth 115 within the retina 122. The depth space image 334 is defined by an axis 336 corresponding to the length 113 of the sample beam 112 and an axis 338 corresponding to the depth 115 into the retina 122. Each pixel value of the first plurality of pixel values indicates an intensity at a particular depth 115 within the retina 122 and at a particular lateral position along the length 113. The depth space image 334 corresponds to the position 142 on the retina 122 along the axis 124.

The computing system 901 can also transform the wavelength space image 140 to generate a depth space image 340 comprising a second plurality of pixel values. The depth space image 340 is defined by the axis 336 and the axis 338. Each pixel value of the second plurality of pixel values indicates an intensity at a particular depth 115 within the retina 122 and a particular lateral position along the length 113. The depth space image 340 corresponds to the position 144 on the retina 122 along the axis 124.

The computing system 901 can also transform the wavelength space image 135 to generate a depth space image 335 comprising a third plurality of pixel values. The depth space image 335 is defined by the axis 336 and the axis 338. Each pixel value of the third plurality of pixel values indicates an intensity at a particular depth 115 within the retina 122 and a particular lateral position along the length 113. The depth space image 335 corresponds to the position 142 on the retina 122 along the axis 124.

The computing system 901 can also transform the wavelength space image 141 to generate a depth space image 341 comprising a fourth plurality of pixel values. The depth space image 341 is defined by the axis 336 and the axis 338. Each pixel value of the fourth plurality of pixel values indicates an intensity at a particular depth 115 within the retina 122 and a particular lateral position along the length 113. The depth space image 341 corresponds to the position 144 on the retina 122 along the axis 124. Thus, wavelength space images can also be used to analyzed the effects that the visible light 128 has on the retina 122.

The computing system 901 is configured to generate a three-dimensional image of the retina 122 by combining the depth space image 334 and the depth space image 340. The computing system 901 is also configured to generate a three-dimensional image of the retina 122 by combining the depth space image 335 and the depth space image 341.

In other embodiments, the wavelength space images 134, 135, 140, and 141 are transformed by the computing system 901 into depth space images 334, 340, 335, and 341 that depict phase of the interference beam 116 corresponding to various positions within the retina 122, instead of intensity of the interference beam 116 corresponding to various positions within the retina 122. The absolute value of the transformed data corresponds to signal intensity of the interference beam 116 whereas the argument of the transformed data corresponds to relative phase of the interference beam 116.

In examples where the depth space images 334, 335, 340, and 341 depict signal phase of the interference beam 116, the computing system 901 can be further configured to use the depth space image 334 to determine a first optical path length 401 that separates a first end 410 of an object (e.g., a retinal neuron) from a second end 411 of the object. Generally, the computing system 901 will use the depth space image 334 to determine a first signal phase difference between the signal phase corresponding to the first end 410 and the signal phase corresponding to the second end 411, and use the first signal phase difference to derive the first optical path length 401. In some examples, the depth space image 334 represents a first time, for example, before the retina 122 is stimulated by the visible light 128. In this context, the first end 410 additionally corresponds to a first intensity peak of a corresponding depth space image representing signal intensity obtained at the first time. The second end 411 additionally corresponds to a second intensity peak of the corresponding depth space image representing signal intensity at the first time. The computing system 901 can also use the depth space image 335 to determine a second optical path length 501 that separates the first end 410 and the second end 411 at a second subsequent time, for example, after the retina 122 is stimulated by the visible light 128. Generally, the computing system 901 will use the depth space image 335 to determine a second signal phase difference between the signal phase corresponding to the first end 410 and the signal phase corresponding to the second end 411, and use the second signal phase difference to derive the second optical path length 501. In this context, the first end 410 additionally corresponds to a third intensity peak of the corresponding depth space image representing signal intensity at the second time. The second end 411 additionally corresponds to a fourth intensity peak of the corresponding depth space image representing signal intensity at the second time. Comparing signal phases in this way can yield very high temporal and spatial resolution when analyzing how the retina reacts to stimuli. In a particular embodiment, the detected change in optical path length of a retinal neuron can represent an actual change in size or shape of the retinal neuron, or a change in physiological composition that changes the optical index of the retinal neuron.

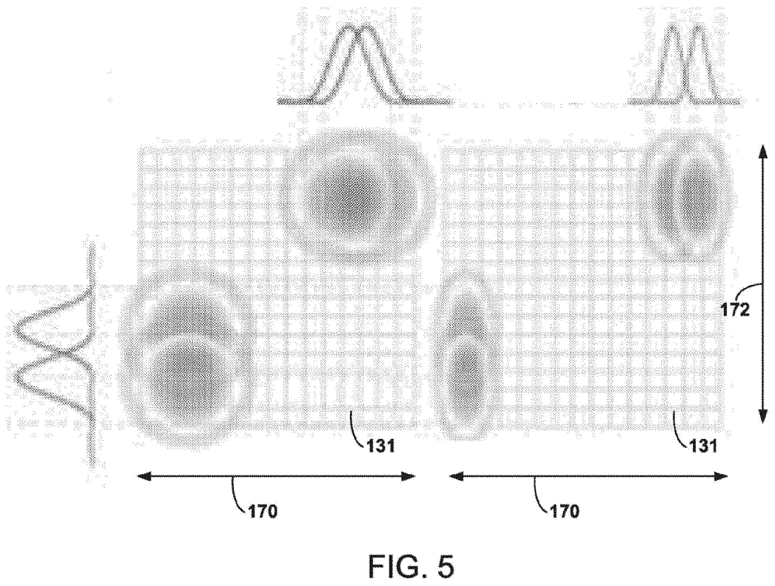

FIG. 5 depicts imaging techniques. For example, the optical module 168 can be used to compress or expand the interference beam 116 independently in the spectral or spatial dimension before the interference beam 116 is dispersed by the dispersive element 132. In a first example, the axis 170 represents the spectral axis of the image sensor 130 and the axis 172 represents the spatial axis of the image sensor 130. Thus, the optical module 168 can be operated to compress the dimension of the interference beam 116 that corresponds to the axis 170 and/or expand the dimension of the interference beam 116 that corresponds to the axis 172, to make efficient use of the area of the image sensor 130. In a second example, the axis 170 represents the spatial axis of the image sensor 130 and the axis 172 represents the spectral axis of the image sensor 130. The ratio of the focal lengths of the cylindrical lenses decides the ratio of the major and minor axes of the ellipses. By reducing the size along the spectrum dimension, better spectral resolution is achievable, without sacrificing spatial resolution along the line dimension.

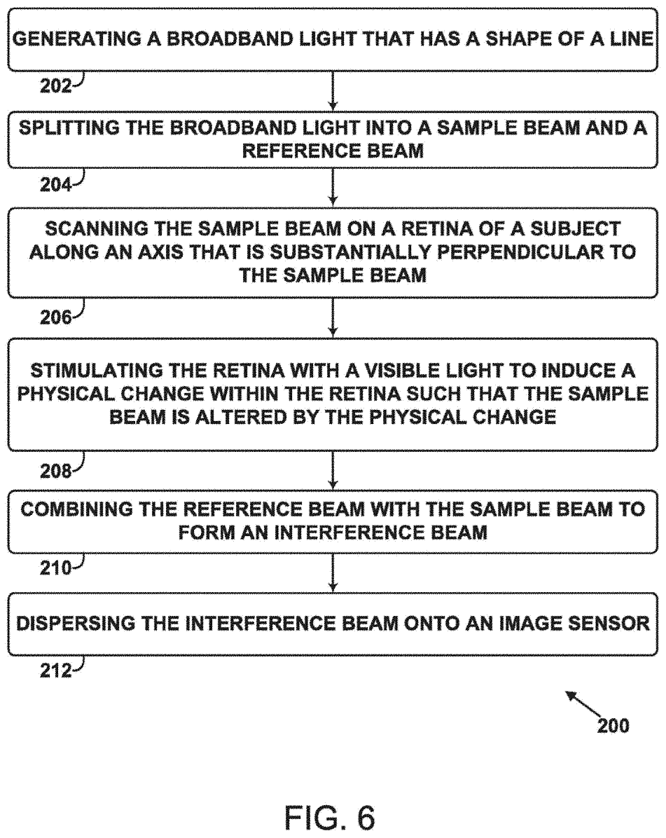

FIG. 6 is a block diagram of a method 200 of operating the optical instrument 100. As shown in FIG. 6, the method 200 includes one or more operations, functions, or actions as illustrated by blocks 202, 204, 206, 208, 210, and 212. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 202, the method 200 includes generating the broadband light 104 that has a shape of the line 108.

At block 204, the method 200 includes splitting the broadband light 104 into the sample beam 112 and the reference beam 114.

At block 206, the method 200 includes scanning the sample beam 112 on the retina 122 of a subject along the axis 124 that is substantially perpendicular to the sample beam 112.

At block 208, the method 200 includes stimulating the retina 122 with the visible light 128 to induce a physical change within the retina 122 such that the sample beam 112 is altered by the physical change.

At block 210, the method 200 includes combining the reference beam 114 with the sample beam 112 to form the interference beam 116.

At block 212, the method 200 includes dispersing the interference beam 116 onto the image sensor 130.

The method 200 can involve non-invasively imaging retinal function in the subject on a cellular scale, detecting a change in size or shape or physiology of a retinal neuron, and/or in-vivo measurement of electrical activity of a/many retinal neuron in the subject. The method 200 can also involve diagnosing a retinal disorder, such as a retinal disorder that affects one or more of photoreceptors, retinal pigment epithelium, choroid, ganglion cells, or a nerve fiber layer, vasculature. The method 200 can also involve determining a physiological composition of a retinal neuron in the subject and determining the change in physiological composition with light stimuli.

The method 200 can also involve treating and/or diagnosing one or more of the following disorders: retinal tear, retinal detachment, diabetic retinopathy, epiretinal membrane, macular hole, wet macular degeneration, dry macular degeneration, retinitis pigmentosa, achromatopsia, and macular telangiectasia.

A retinal tear occurs when the vitreous shrinks and tugs on the retina with enough traction to cause a break in the tissue. A retinal tear is often accompanied by symptoms such as floaters and flashing lights.

Retinal detachment typically occurs in the presence of fluid under the retina. This usually occurs when fluid passes through a retinal tear, causing the retina to lift away from the underlying tissue layers.

Diabetic retinopathy generally involves capillary fluid leakage and/or abnormal capillary development and bleeding into and under the retina, causing the retina to swell, which can blur or distort vision.

Epiretinal membrane generally involves the development of a tissue-like scar or membrane that pulls up on the retina, which distorts vision. Objects may appear blurred or crooked.

Macular hole typically involves a small defect in the center of the retinal macula, which may develop from abnormal traction between the retina and the vitreous, or it may follow an injury to the eye.

Macular degeneration generally involves retinal macula deterioration, causing symptoms such as blurred central vision or a blind spot in the center of the visual field. There are two types—wet macular degeneration and dry macular degeneration. Many people will first have the dry form characterized by the presence of drusen that can distort vision, which can progress to the wet form in one or both eyes, characterized by blood vessel formation under the macula which can bleed and lead to severe vision effects including permanent loss of central vision.

Retinitis pigmentosa is an inherited degenerative disease affecting the retina that causes loss of night and side vision. Retinitis pigmentosa is typically characterized by a breakdown or loss of cells in the retina.

While various example aspects and example embodiments have been disclosed herein, other aspects and embodiments will be apparent to those skilled in the art. The various example aspects and example embodiments disclosed herein are for purposes of illustration and are not intended to be limiting, with the true scope and spirit being indicated by the following claims.

1. An optical instrument comprising:
a first light source configured to generate a broadband light;
an optical module configured to collimate the broadband light and focus the broadband light into a line;
a beam splitter configured to split the broadband light into a sample beam and a reference beam and configured to combine the reference beam with the sample beam to form an interference beam;
a control system configured to scan the sample beam on a retina of a subject along an axis that is substantially perpendicular to the sample beam;
a second light source configured to stimulate the retina with a visible light to induce a physical change within the retina such that the sample beam is altered by the physical change;
an image sensor; and
a dispersive element configured to receive the interference beam from the beam splitter and to disperse the interference beam onto the image sensor.

2. The optical instrument of claim 1, wherein the axis is a first axis, wherein the image sensor is configured to:
capture a first wavelength space image of the interference beam after the interference beam has been dispersed by the dispersive element, the first wavelength space image being defined by a second axis that corresponds to a length of the sample beam and a third axis that corresponds to wavelengths of the sample beam, the first wavelength space image corresponding to a first position on the retina along the first axis; and
capture a second wavelength space image of the interference beam after the interference beam has been dispersed by the dispersive element, the second wavelength space image being defined by the second axis and the third axis, the second wavelength space image corresponding to a second position on the retina along the first axis.

3. The optical instrument of claim 2, further comprising a computing system that is configured to:
transform the first wavelength space image to generate a first depth space image comprising a first plurality of pixel values, the first depth space image defined by a fourth axis corresponding to the length of the sample beam and a fifth axis corresponding to depth into the retina, each pixel value of the first plurality of pixel values indicating an intensity at a depth within the retina and a lateral position on the retina that corresponds to the pixel value of the first plurality, the first depth space image corresponding to the first position on the retina along the first axis; and
transform the second wavelength space image to generate a second depth space image comprising a second plurality of pixel values, the second depth space image defined by the fourth axis and the fifth axis, each pixel value of the second plurality of pixel values indicating an intensity at a depth within the retina and a lateral position on the retina that corresponds to the pixel value of the second plurality, the second depth space image corresponding to the second position on the retina along the first axis.

4. (canceled)

5. The optical instrument of claim 3, the computing system being further configured to, subsequent to capturing the first wavelength space image and the second wavelength space image:
capture a third wavelength space image of the interference beam after the interference beam has been dispersed by the dispersive element, the third wavelength space image being defined by the second axis and the third axis, the third wavelength space image corresponding to the first position on the retina along the first axis; and
capture a fourth wavelength space image of the interference beam after the interference beam has been dispersed by the dispersive element, the fourth wavelength space image being defined by the second axis and the third axis, the fourth wavelength space image corresponding to the second position on the retina along the first axis;
transform the third wavelength space image to generate a third depth space image comprising a third plurality of pixel values, the third depth space image defined by the fourth axis and the fifth axis, each pixel value of the third plurality of pixel values indicating an intensity at a depth within the retina and a lateral position on the retina that corresponds to the pixel value of the third plurality, the third depth space image corresponding to the first position on the retina along the first axis; and
transform the fourth wavelength space image to generate a fourth depth space image comprising a fourth plurality of pixel values, the fourth depth space image defined by the fourth axis and the fifth axis, each pixel value of the fourth plurality of pixel values indicating an intensity at a depth within the retina and a lateral position on the retina that corresponds to the pixel value of the fourth plurality, the fourth depth space image corresponding to the second position on the retina along the first axis.

6-7. (canceled)

8. The optical instrument of claim 2, further comprising a computing system that is configured to:
transform the first wavelength space image to generate a first depth space image comprising a first plurality of pixel values, the first depth space image defined by a fourth axis corresponding to the length of the sample beam and a fifth axis corresponding to depth into the retina, each pixel value of the first plurality of pixel values indicating a phase at a depth within the retina and a lateral position on the retina that corresponds to the pixel value of the first plurality, the first depth space image corresponding to the first position on the retina along the first axis; and
transform the second wavelength space image to generate a second depth space image comprising a second plurality of pixel values, the second depth space image defined by the fourth axis and the fifth axis, each pixel value of the second plurality of pixel values indicating a phase at a depth within the retina and a lateral position on the retina that corresponds to the pixel value of the second plurality, the second depth space image corresponding to the second position on the retina along the first axis.

9. (canceled)

10. The optical instrument of claim 8, the computing system being further configured to, subsequent to capturing the first wavelength space image and the second wavelength space image:
capture a third wavelength space image of the interference beam after the interference beam has been dispersed by the dispersive element, the third wavelength space image being defined by the second axis and the third axis, the third wavelength space image corresponding to the first position on the retina along the first axis; and
capture a fourth wavelength space image of the interference beam after the interference beam has been dispersed by the dispersive element, the fourth wavelength space image being defined by the second axis and the third axis, the fourth wavelength space image corresponding to the second position on the retina along the first axis;
transform the third wavelength space image to generate a third depth space image comprising a third plurality of pixel values, the third depth space image defined by the fourth axis and the fifth axis, each pixel value of the third plurality of pixel values indicating a phase at a depth within the retina and a lateral position on the retina that corresponds to the pixel value of the third plurality, the third depth space image corresponding to the first position on the retina along the first axis; and
transform the fourth wavelength space image to generate a fourth depth space image comprising a fourth plurality of pixel values, the fourth depth space image defined by the fourth axis and the fifth axis, each pixel value of the fourth plurality of pixel values indicating a phase at a depth within the retina and a lateral position on the retina that corresponds to the pixel value of the fourth plurality, the fourth depth space image corresponding to the second position on the retina along the first axis.

11-12. (canceled)

13. The optical instrument of claim 1, wherein the axis is a first axis, the optical instrument further comprising a computing system configured to:
capture a first wavelength space image of the interference beam after the interference beam has been dispersed, the first wavelength space image being defined by a second axis that corresponds to a length of the sample beam and a third axis that corresponds to wavelengths of the sample beam, the first wavelength space image corresponding to a first position on the retina along the first axis and a first time;
capture a second wavelength space image of the interference beam after the interference beam has been dispersed, the second wavelength space image being defined by the second axis and the third axis, the second wavelength space image corresponding to the first position on the retina along the first axis and a second time that is subsequent to the first time;
transform the first wavelength space image to generate a first depth space image of the interference beam after the interference beam has been dispersed, the first depth space image defined by a fourth axis corresponding to the length of the sample beam and a fifth axis corresponding to depth into the retina, each pixel value of the first depth space image indicating a phase at a depth within the retina and a lateral position on the retina, the first depth space image corresponding to the first position on the retina along the first axis and the first time;
transform the second wavelength space image to generate a second depth space image of the interference beam after the interference beam has been dispersed, the second depth space image defined by the fourth axis and the fifth axis, each pixel value of the second depth space image indicating a phase at a depth within the retina and a lateral position on the retina, the second depth space image corresponding to the first position on the retina along the first axis and the second time;
use the first depth space image to determine a first signal phase difference between a first end of an object and a second end of the object, wherein the first end corresponds to a first intensity peak of another depth space image and the second end corresponds to a second intensity peak of the other depth space image, wherein the first signal phase difference corresponds to a first optical path length; and
use the second depth space image to determine a second signal phase difference between the first end and the second end, wherein the first end corresponds to a first intensity peak of an additional depth space image and the second end corresponds to a second intensity peak of the additional depth space image, wherein the second signal phase difference corresponds to a second optical path length.

14-28. (canceled)

29. The optical instrument of claim 1, further comprising a second optical module configured to modify the interference beam after the interference beam has been dispersed by the dispersive element to increase spatial resolution of the interference beam and decrease spectral resolution of the interference beam.

30. The optical instrument of claim 1, further comprising a second optical module configured to modify the interference beam after the interference beam has been dispersed by the dispersive element to decrease spatial resolution of the interference beam and increase spectral resolution of the interference beam.

31-39. (canceled)

40. The optical instrument of claim 1, further comprising a computing system configured to:
capture, over a first period of time, a first wavelength space image of the interference beam after the interference beam has been dispersed and a second wavelength space image of the interference beam after the interference beam has been dispersed, the first wavelength space image corresponding to a first position along the axis and the second wavelength space image corresponding to a second position along the axis;
capture, over a second period of time that is subsequent to the first period of time, a third wavelength space image of the interference beam after the interference beam has been dispersed and a fourth wavelength space image of the interference beam after the interference beam has been dispersed, the third wavelength space image corresponding to the first position along the axis and the fourth wavelength space image corresponding to the second position along the axis;
transform the first wavelength space image to generate a first depth space image of the interference beam after the interference beam has been dispersed, each pixel value of the first depth space image indicating a signal phase at a respective location within the retina;
transform the second wavelength space image to generate a second depth space image of the interference beam after the interference beam has been dispersed, each pixel value of the second depth space image indicating a signal phase at a respective location within the retina;
transform the third wavelength space image to generate a third depth space image of the interference beam after the interference beam has been dispersed, each pixel value of the third depth space image indicating a signal phase at a respective location within the retina;
transform the fourth wavelength space image to generate a fourth depth space image of the interference beam after the interference beam has been dispersed, each pixel value of the fourth depth space image indicating a signal phase at a respective location within the retina;
use the first depth space image and the third depth space image to determine a first signal phase difference between a first signal phase corresponding to a first retinal feature during the first period of time and a second signal phase corresponding to the first retinal feature during the second period of time; and
use the second depth space image and the fourth depth space image to determine a second signal phase difference between a third signal phase corresponding to a second retinal feature during the first period of time and a fourth signal phase corresponding to the second retinal feature during the second period of time.

41. The optical instrument of claim 1, further comprising a computing system configured to:
capture, via the image sensor, a first wavelength space image of the interference beam after the interference beam has been dispersed;
transform the first wavelength space image to generate a first depth space image of the interference beam after the interference beam has been dispersed, each pixel value of the first depth space image indicating a signal phase at a particular position within the retina;
transform the first wavelength space image to generate a second depth space image of the interference beam after the interference beam has been dispersed, each pixel value of the second depth space image indicating a signal intensity at the particular position within the retina;
identify a first intensity of the of the second depth space image and a second intensity of the second depth space image, the first intensity corresponding to a first retinal feature and the second intensity corresponding to a second retinal feature; and
use the first depth space image to determine a first signal phase difference between a first signal phase corresponding to the first retinal feature and a second signal phase corresponding to the second retinal feature.

42. The optical instrument of claim 1, further comprising a computing system configured to:
capture, via the image sensor, a first wavelength space image of the interference beam after the interference beam has been dispersed;
transform the first wavelength space image to generate a first depth space image of the interference beam after the interference beam has been dispersed, each pixel value of the first depth space image indicating a signal intensity at a particular position within the retina;
identify a first intensity of the of the first depth space image and a second intensity of the first depth space image, the first intensity corresponding to a first retinal feature and the second intensity corresponding to a second retinal feature; and
determine a distance between the first retinal feature and the second retinal feature.

43. A method of operating an optical instrument, the method comprising:
generating a broadband light that has a shape of a line;
splitting the broadband light into a sample beam and a reference beam;
scanning the sample beam on a retina of a subject along an axis that is substantially perpendicular to the sample beam;
stimulating the retina with a visible light to induce a physical change within the retina such that the sample beam is altered by the physical change;
combining the reference beam with the sample beam to form an interference beam; and
dispersing the interference beam onto an image sensor.

44-78. (canceled)

79. The method of claim 43, wherein the method comprises detecting a change in size or shape of a retinal neuron.

80. The method of claim 43, wherein the method comprises in-vivo measurement of electrical activity of a retinal neuron in the subject.

81. The method of claim 43, wherein the method is performed to diagnose or treat a retinal disorder.

82. The method of claim 81, wherein the retinal disorder affects one or more of photoreceptors, retinal pigment epithelium, choroid, ganglion cells, or a nerve fiber layer.

83. The method of claim 81, wherein the retinal disorder is selected from the group consisting of retinal tear, retinal detachment, diabetic retinopathy, epiretinal membrane, macular hole, wet macular degeneration, dry macular degeneration, and retinitis pigmentosa.

84. The method of claim 43, further comprising determining a physiological composition of a retinal neuron in the subject.

85-91. (canceled)

92. A non-transitory computer readable medium storing instructions that, when executed by one or more processors of an optical instrument, cause the optical instrument to perform functions comprising:
generating a broadband light that has a shape of a line;
splitting the broadband light into a sample beam and a reference beam;
scanning the sample beam on a retina of a subject along an axis that is substantially perpendicular to the sample beam;
stimulating the retina with a visible light to induce a physical change within the retina such that the sample beam is altered by the physical change;
combining the reference beam with the sample beam to form an interference beam; and
dispersing the interference beam onto an image sensor.
